(19) United States
(12) Patent Application Publication
(10) Pub. No.: US 2026/0000312 A1
(43) Pub. Date: Jan. 1, 2026

(54) MOUTHPIECE MANUFACTURING METHOD

(71) Applicant: James R. Glidewell Dental Ceramics, Inc., Newport Beach, CA (US)

(72) Inventors: Santosh Gaikwad, Irvine, CA (US); Fernando Vera, Fountain Valley, CA (US); Thomas Quinn, Ware (GB)

(73) Assignee: James R. Glidewell Dental Ceramics, Inc., Newport Beach, CA (US)

(21) Appl. No.: 19/252,608

(22) Filed: Jun. 27, 2025

Related U.S. Application Data
(60) Provisional application No. 63/666,500, filed on Jul. 1, 2024.

(51) Int. Cl. A61B 5/024; A61B 5/00; A61B 5/1455; B29C 51/12; B29C 51/46; B29L 31/00

(52) U.S. Cl. CPC A61B 5/02427; A61B 5/14552; A61B 5/6803; A61B 2562/12; B29C 51/12; B29C 51/46; B29L 2031/753

(57) ABSTRACT

A method for manufacturing a mouthpiece is disclosed. The method includes receiving a digital representation of a teeth model, creating a shell model overlying an outer surface of the teeth model, identifying a target position on the shell model where a sensor of the mouthpiece is to be placed, and generating a modified teeth model comprising a dimple underneath the shell model at the target position.

### CROSS-REFERENCE TO RELATED APPLICATIONS

This application claims the benefit of, and priority to, the earlier filing date of U.S. Provisional Application No. 63/666,500, filed Jul. 1, 2024, which is incorporated herein by reference in its entirety.

### FIELD

The present disclosure concerns mouthpieces, as well as systems and methods for manufacturing the same.

### BACKGROUND

Fitness tracking devices, or fitness trackers, have been used to monitor fitness-related metrics and provide feedback on an individual's performance. Exemplary metrics monitored by the fitness trackers include heart rate, respiration rate, oxygen saturation level, etc. Many existing fitness trackers are cumbersome and/or sensitive to motion of the users. For example, some fitness trackers include heart rate monitors which place electrodes or a strap around the user's chest. A less intrusive technique, pulse oximetry, uses a photoplethysmograph (PPG) sensor to non-invasively measure light absorption through a user's tissue to determine heart rate and oxygen saturation level. However, this requires the user to remain relatively motionless to obtain a good signal. Thus, there is a room for improvement in wearable technology for fitness tracking.

### SUMMARY

Described herein are mouthpieces configured to measure physiological metrics of users who wear the mouthpieces. Methods for manufacturing the mouthpieces are also disclosed.

Certain examples of the disclosure concern a method for manufacturing a mouthpiece. The method includes receiving a digital representation of a teeth model, creating a shell model overlying an outer surface of the teeth model, identifying a target position on the shell model where a sensor of the mouthpiece is to be placed, and generating a modified teeth model comprising a dimple underneath the shell model at the target position.

Certain examples of the disclosure also concern a system for manufacturing a mouthpiece. The system includes memory, one or more hardware processors coupled to the memory, and one or more computer readable storage media storing instructions that, when loaded into the memory, cause the one or more hardware processors to perform operations. The operations include receiving a digital representation of a teeth model, creating a shell model overlying an outer surface of the teeth model, identifying a target position on the shell model where a sensor of the mouthpiece is to be placed, and generating a modified teeth model comprising a dimple underneath the shell model at the target position.

Certain examples of the disclosure further concern a mouthpiece. The mouthpiece includes a first elastomeric layer configured to be placed over a user's teeth and gum, a second elastomeric layer covering the first elastomeric layer, and an electronic circuit sandwiched between the first elastomeric layer and the second elastomeric layer. The first elastomeric layer includes a protruded portion configured to press against a palate of the user when the mouthpiece is worn by the user. The electronic circuit includes a sensor placed in a pocket formed by the protruded portion.

The mouthpiece described herein can be any devices mounted on user's teeth, such as a mouthguard, a gum shield, an orthodontic appliance (e.g., used to alleviate or treat snoring or sleep apnea), a retainer, among others.

The foregoing and other features and advantages of the disclosed technologies will become more apparent from the following detailed description, which proceeds with reference to the accompanying figures.

### BRIEF DESCRIPTION OF THE DRAWINGS

FIG. 1A is a schematic cross-sectional view of a mouthpiece.

FIG. 1B schematically depicts a circuit board embedded within the mouthpiece of FIG. 1A.

FIG. 2 is an overall block diagram of a mouthpiece manufacturing system.

FIG. 3 is a flowchart illustrating an example overall method of manufacturing a mouthpiece with an embedded circuit board.

FIG. 4A is an example digital representation of a teeth model.

FIG. 4B depicts a gap-filled teeth model.

FIG. 5A is a front elevation view of a shell model generated based on the teeth model of FIG. 4B.

FIG. 5B is a perspective view of the shell model of FIG. 5A with added objects representing different parts of a circuit board.

FIG. 6A is a cross-sectional view of the shell model of FIG. 5B and the underlying the teeth model.

FIG. 6B is a cross-sectional view of a modified teeth model including a dimple.

FIG. 7A is a top perspective view of a mold created based on the modified teeth model of FIG. 6B.

FIG. 7B depicts a first layer of a mouthpiece formed over the mold of FIG. 7A.

FIG. 8A depicts a circuit board having a sensor placed over the first layer of FIG. 7B.

FIG. 8B depicts a second layer of the mouthpiece formed over the first layer and the circuit board of FIG. 8B.

FIG. 8C schematically depicts a cross-section of the mouthpiece of FIG. 8B at the location of the sensor.

FIG. 9 is a block diagram of an example computing system in which described technologies can be implemented.

### DETAILED DESCRIPTION

### Overview of Smart Mouthpiece Technologies

To overcome problems of many existing fitness trackers, smart mouthpieces have been developed. Specifically, a mouthpiece can be customized to fit a user's teeth so that the mouthpiece can be worn, in use, within a user's mouth. The mouthpiece can be embedded with a printed circuit board (“PCB”, or simply “circuit board”) with one or more sensors configured to measure the user's physiological signals. The mouthpiece with integrated sensing capabilities can outperform many existing fitness trackers worn on other body parts. For example, its personalized fit ensures stability and minimal interference from body movements, leading to more accurate, noise-free data. This allows for precise, real-time monitoring of physiological signals, even during intense activity, without the discomfort or inconvenience associated with chest straps or wristbands.

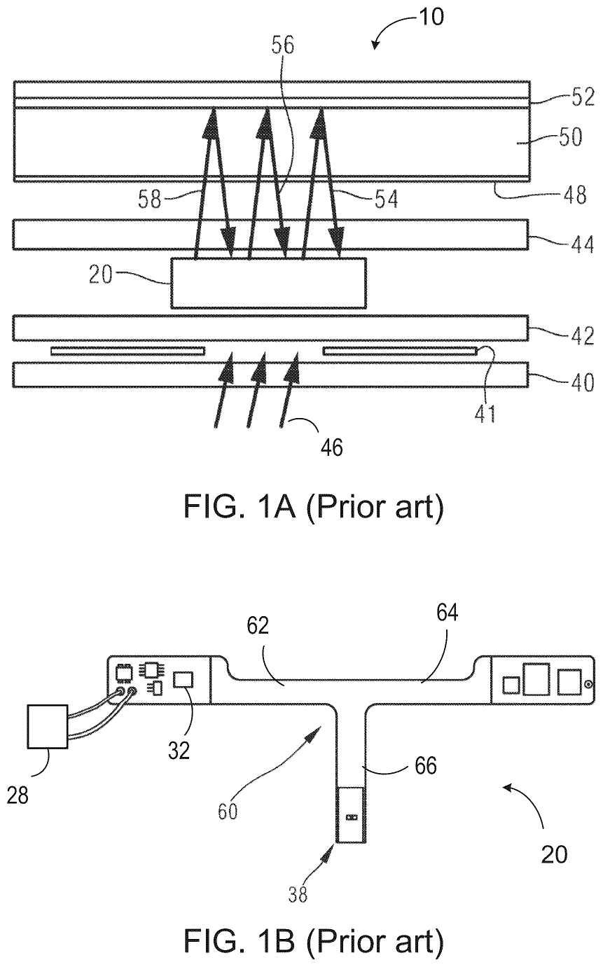

FIG. 1A depicts an example mouthpiece 10 having an embedded circuit board 20, and FIG. 1B depicts parts of the circuit board 20.

As shown in FIG. 1A, the mouthpiece 10 can include multiple thin layers of material. An outer layer 40 (also referred to as “protective layer”) can be substantially transparent to allow light to pass therethrough. An inner layer 44 that is closest to the user's teeth and gum can be substantially transparent to allow light to pass therethrough. A mid-layer 42, which is positioned between the inner layer 44 and the outer layer 40, can be opaque or have a very dark color (e.g., black) that substantially prevents light from passing therethrough. Each of the outer layer 40, mid-layer 42, and inner layer 44 can be formed of Ethylene-Vinyl Acetate (EVA) plastic through a thermoforming process. In some examples, labels 41 carrying information and/or logos may be sandwiched between the outer layer 40 and the mid-layer layer 42.

The circuit board 20, which includes a light source 32 (e.g., an LED) and a PPG sensor 38, can be substantially sandwiched between the mid-layer 42 and the inner layer 44. Thus, any ambient light 46 entering the user's mouth can be blocked by the mid-layer 42 and thus prevented from interfering with the PPG sensor 38. Light emitted from the light source 32 (e.g., with wavelength between 10 nm and 3000 nm) can shine through the inner layer 44, onto the user's skin 48, and through an underlying mouth tissue 50 to blood vessel(s) 52. The reflected light can return to the PPG sensor 38, where the amplitudes of reflected green light 54, red light 56 and infrared light 58 can be measured, based on which the user's PPG signal can be obtained. Physiological metrics such as heart rate, oxygen concentration, respiration rate, etc., can be obtained from the PPG signal. In some examples, the PPG sensor 38 can be placed adjacent to the user's palate, which can reflect the light emitted by the light source 32 to the PPG sensor 38.

As shown in FIG. 1B, the circuit board 20 can be configured to have multiple separate sections, connected together by a flexible connector in the form of, for example, polyimide flexible PCB material. In the depicted example, the circuit board 20 has a left arm 62, a right arm 64, and a central leg 66 that are interconnected with each other to define a substantially T-shaped flexible connector 60. Some electronic components of the circuit board 20 (e.g., the light source 32, external flash memory, etc.) can be disposed at an end portion of the left arm 62. A power supply unit 28 (e.g., a Lithium polymer battery) can also be connected to the left arm 62 via conducting wires or cables. Some electronic components of the circuit board 20 (e.g., a microcontroller, an inertial measuring unit, a high impact accelerometer, etc.) can be disposed at an end portion of the right arm 64. In the depicted example, the PPG sensor 38 can be disposed at an end portion of the central leg 66. It should be understood that the shape and/or size of the circuit board 20 can vary, and the exact locations of different electronic components of the circuit board 20 can also differ from the example depicted in FIG. 1B. For example, some electronic components disposed on the left arm 62 can be switched to the right arm 64, or vice versa.

In some circumstances, the mouthpiece 10 can be paired with a case insert. When not in use, the mouthpiece 10 can be placed within the case insert for storage. Optionally, the case insert can include a wireless charging system configured to wirelessly charge the power supply unit 28 of the mouthpiece 10. For example, the case insert can include one or more transmitting coils that are positioned to align with a receiving coil located on the circuit board 20 of the mouthpiece 10 so as to enable wireless charging of the power supply unit 28 through magnetic coupling between the transmitting and receiving coils.

Further details of the mouthpieces with embedded circuit boards are described in U.K. Patent Application GB 2595723, which is incorporated by reference herein. Additional details of the case insert and methods for making the case insert are described in U.S. Provisional Application No. 63/621,569, which is also incorporated by reference herein.

Proper placement the PPG sensor 38 within the mouthpiece 10 can be critical for achieving high signal-to-noise ratio of the PPG signal. As described above, one desirable location for measuring the PPG signal is the user's palate. When assembling the mouthpiece, an operator needs to find a proper location on the inner layer 44 to place the PPG sensor 38 such that when fully assembled, the PPG sensor 38 sits on top of a flat portion of the user's palate to ensure reliable PPG measurement.

However, this task can be challenging in certain circumstances. The uneven topology of the palate means that the sensor must be positioned with precision to maintain consistent contact. Any gaps or misalignment can result in signal degradation, which can be further exacerbated by the natural moisture and movement within the mouth during use. The operator, however, may inadvertently place the sensor over a contoured part of the inner layer which does not align with the flat portion of the palate. Moreover, the variability in palate shapes across different users adds another layer of complexity, requiring a customized approach to sensor placement for each individual mouthpiece. This necessity for customization can lead to increased production time and costs, as well as requiring operators to have a higher level of skill and training to identify the optimal sensor location. Additionally, the pressure exerted by the mouthpiece on the sensor needs to be carefully calibrated; while too much pressure can cause discomfort to the user, too little may result in a loose fit, leading to inaccurate readings.

Many of the above challenges can be overcome by the technologies described herein, which allow an operator to place the PPG sensor easily and precisely within the mouthpiece to ensure its effective functioning.

### Example System for Manufacturing Mouthpieces

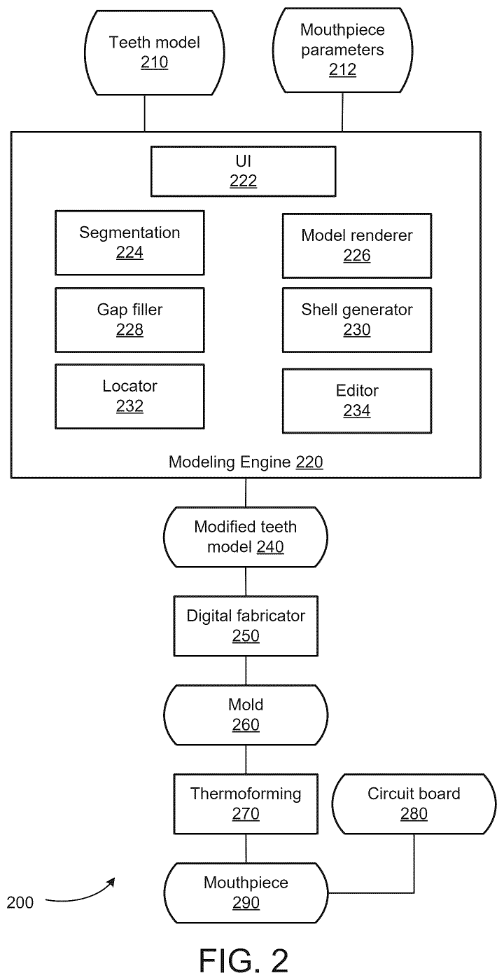

FIG. 2 shows an overall block diagram of an example manufacturing system 200 that can be used for computer-aided design (CAD) and computer-aided manufacturing (CAM) of smart mouthpieces, such as the mouthpiece 10 described above.

The manufacturing system 200 includes a modeling engine 220 which is configured to receive a digital representation of a user-specific teeth model 210 (or simply “teeth model”) and specific parameters 212 of a desired mouthpiece 290, and to generate a modified teeth model 240.

The modified teeth model 240, typically represented as CAD files, can be supplied to a digital fabricator 250 to produce a mold 260 for the mouthpiece. The digital fabricator 250 could be a 3D printer, a computer numerical control (CNC) machine, a laser cutter, or any other computer-controlled manufacturing devices set up to execute a CAM process.

A thermoforming apparatus 270 is configured to create the mouthpiece 290 based on the mold 260. The thermoforming apparatus 270 is a machine or equipment configured to shape plastic materials into complex forms, e.g., by heating a thermoplastic sheet until it becomes pliable, then stretching it over a mold (e.g., the mold 260) where it is shaped by vacuum pressure or mechanical force. Once the material conforms to the mold, it is cooled and retains the imposed shape, which can be further processed like trimming to its final form.

Like the mouthpiece 10 described above, the mouthpiece 290 includes at least two layers of elastomeric materials. Similarly, a prefabricated circuit board 280 (similar to the circuit board 20) having a PPG sensor can be embedded between these two elastomeric layers of the mouthpiece 290. Positioning of the PPG sensor within the mouthpiece 290 can be precisely controlled due to a dimple formed on the modified teeth model 240, as described more fully below. This ensures that the PPG sensor can maintain optimal alignment with the user's palate for accurate readings.

The manufacturing process, from receiving the teeth model 210 and mouthpiece parameters 212, to generating the modified teeth model 240 and fabricating the mold 260, can be automated or substantially automated, thanks to the capabilities of the modeling engine 220 and the digital fabricator 250. In some cases, the entire manufacturing process, including the step for generating the final mouthpiece 290, can also be automated, e.g., by integrating the thermoforming apparatus 270 with a computerized control system.

As shown in FIG. 2, the modeling engine 220 can include a plurality of components, including a user interface 222 (UI), a segmentation unit 224, a model renderer 226, a gap filler 228, a shell generator 230, a locator 232, and an editor 234. Each of these components is configured to implement one or more functions described further below. In other examples, some of these components can be combined and/or one or more of these components may be split into multiple sub-components.

The modeling engine 220 can receive the teeth model 210 and mouthpiece parameters 212 through the user interface 222. The teeth model 210 can be represented by 3D model files in a variety of data format, such as STL (Standard Tessellation Language), PLY (Polygon File Format), OBJ (Object File Format), DICOM (Digital Imaging and Communications in Medicine), STEP (Standard for the Exchange of Product Data), etc. The mouthpiece parameters 212 define various geometric parameters of the mouthpiece, which can be fabricated based on the same or substantially the same teeth model 210. For example, for the mouthpiece 10 described above, the mouthpiece parameters 212 include, but are not limited to, thicknesses of the inner layer 44, the mid-layer 42, and the outer layer 40, positions and sizes of the circuit board 20 (including the left arm 62, the right arm 64, the central leg 66, etc.) relative to the inner layer 44, positions, shapes, and sizes of various components (e.g., the power supply unit 28, the PPG sensor 38, etc.) located on or being connected to the circuit board 20, etc.

Through the user interface 222, an operator can view and interact with any models rendered by the model renderer 226, e.g., changing viewing angles, zooming in/out, viewing cross-sections, etc. Additionally, through the user interface 222, the operator can also change or edit parameters of the modeling engine 220 that control various aspects of the CAD process (e.g., modeling rendering, segmentation, shell generation, etc.), as described further below.

The model renderer 226 is configured for displaying the received teeth model 210 and any generated models (e.g., the shell model generated by the shell generator 230 and the modified teeth model 240) on the user interface 222. For a given model, the model renderer 226 can render it into a visual format that can be easily interpreted by the operator, who can view and/or manipulate the rendered model through the user interface 222.

The segmentation unit 224 can be configured for pre-processing the teeth model 210, e.g., by employing segmentation techniques like thresholding, region growing, or watershed to divide the teeth model 210 into distinct objects. These objects can include individual teeth, gum, palate, and more. The segmentation unit 224 can also identify different surfaces of the teeth such as occlusal surface, front surface, lingual surface, etc. As described herein, the occlusal surface refers to the surface of a tooth that comes in contact with the corresponding tooth in the opposite jaw during occlusion or biting, the front surface (also referred to as “facial surface”) refers to the surface of a tooth that faces lip or cheek, and lingual surface refers to the surface of a tooth that faces the tongue. In some examples, the segmentation unit 224 can be configured to implement teeth segmentation using neural networks, as described in U.S. Pat. No. 11,842,484, which is incorporated by reference herein. Other segmentation techniques can also be used by the segmentation unit 224.

The gap filler 228 is configured to identify gaps between adjacent teeth in the teeth model 210 and fill those gaps using virtual fillers. The resulting teeth model can be referred to as a gap-filled teeth model. These virtual fillers can be generated based on the surrounding teeth so that the filled gaps blend seamlessly with the rest of the teeth model, resulting in a continuous teeth structure.

The shell generator 230 is configured to generate a shell model based on the teeth model 210 which is devoid of gaps between teeth (e.g., by applying the gap filler 228 to the teeth model). The shell model represents an inner layer (e.g., the inner layer 44) of the mouthpiece. For example, the size and overall shape of the shell model can match those of the inner layer. The thickness of the inner layer (e.g., specified in the mouthpiece parameters 212) can define a thickness of the shell model. The shell generator 230 can create the shell model using surface offset techniques such as point-based offsetting method of polygonal meshes (where each point on the surface of a polygonal mesh is moved or offset to create a new surface), voxel-based surface offsetting (where the surface of an object, represented as a 3D grid of voxels, is expanded or contracted to create a new surface), or the like.

The locator 232 is configured to identify specific locations on the teeth model and/or the shell model with precision. The editor 234 can be configured to insert or add different objects to the created shell model. For example, the locator 232 can locate a midline of the shell model, based on which can further locate areas on the shell model where objects representing different parts of the circuit board can be placed, e.g., by the editor 234. Locations, sizes, and shapes of those areas can be obtained from mouthpiece parameters 212.

Additionally, the editor 234 can be configured to subtract a selected portion from any of the rendered models. For example, the editor 234 can be configured to remove a portion from the teeth model at a target location (e.g., identified by the locator 232) so as to create a dimple on the teeth model, thereby generating the modified teeth model 240. The shape and size of the removed portion can generally match those of a PPG sensor embedded in the mouthpiece, as described more fully below.

The described manufacturing system 200 can be networked via wired or wireless network connections, including the Internet. Alternatively, the manufacturing system 200 can be connected through an intranet connection (e.g., in a corporate environment, government environment, or the like).

The manufacturing system 200 and any of the other systems described herein can be implemented in conjunction with any of the hardware components described herein, such as the computing systems described below (e.g., processing units, memory, and the like). In any of the examples herein, the teeth model, mouthpiece parameters, the shell model, and the like can be stored in one or more computer-readable storage media or computer-readable storage devices. The technologies described herein can be generic to the specifics of operating systems or hardware and can be applied in any variety of environments to take advantage of the described features.

### Example Methods for Manufacturing Mouthpieces

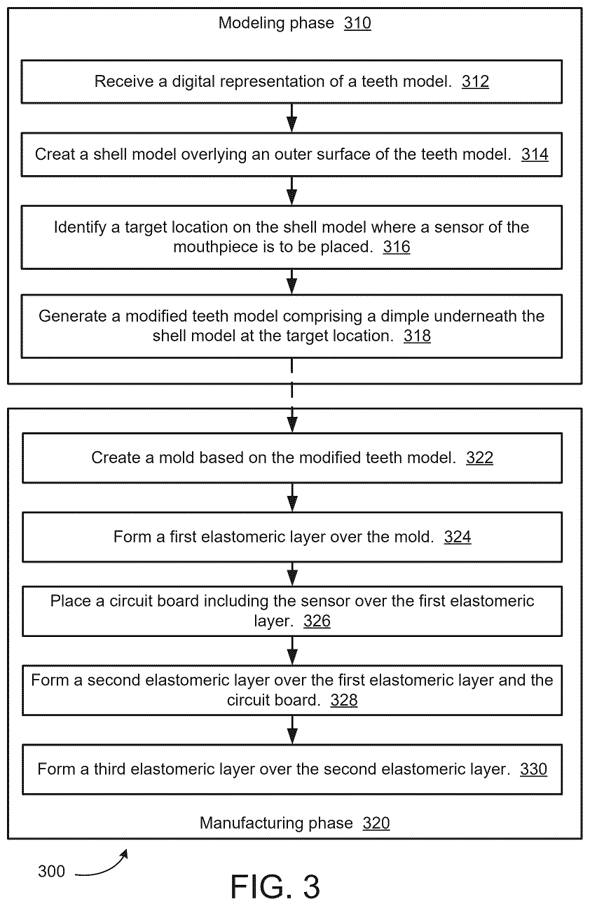

FIG. 3 is a flowchart describing an overall method 300 for manufacturing a mouthpiece having an embedded circuit board. The method 300 can be performed, for example, by using the manufacturing system 200 of FIG. 2.

The method 300 can include two phases: a modeling phase 310 and a manufacturing phase 320. In some examples, the modeling phase 310 can be substantially performed by the modeling engine 220 of FIG. 2, and the manufacturing phase 320 can be substantially performed by the digital fabricator 250 and the thermoforming apparatus 270 of FIG. 2.

At step 312, a digital representation of a teeth model is received. Additionally, parameters of the mouthpiece can also be received as input. The teeth model, which can be specific to a user of the mouthpiece, can be generated by any known imaging modalities. For example, the teeth model can be generated by scanning the user's mouth using an intraoral scanner, or by scanning a traditional impression or model with a desktop optical scanner. The scanned images can then be processed to create a 3D representation of the teeth model. Additionally, X-ray computed tomography (CT) scans can also be used to generate a 3D image of the teeth, which can then be converted into the teeth model.

At step 314, a shell model can be created, e.g., by the shell generator 230, based at least in part on the received teeth model. The shell model is configured to overly an outer surface of the teeth model. The shell model contacts and covers the front or facial side of the teeth model, just like the inner layer of the mouthpiece contacts the front surface of the teeth. The shell model can also include a portion corresponding to a palate of the user.

In some examples, pre-processing of the teeth model can be performed prior to creation of the shell model. For example, one or more gaps between adjacent teeth in the teeth model can be identified and filled with virtual fillers, e.g., by the gap filler 228. Filling gaps between adjacent teeth in the teeth model before generating the first shell model can ensure a smooth and continuous surface for the shell model.

Generally, the size, contour, and thickness of the shell model can match those of the inner layer of the mouthpiece. In other words, the created shell model can be a digital representation of the mouthpiece's inner layer.

At step 316, the method can identify a target position on the shell model where a sensor of the mouthpiece is to be placed. In some examples, the sensor can be a PPG sensor (e.g., the PPG sensor 38). In some examples, identifying the target position on the shell model can include determining a flat surface along a midline of the shell model.

To identify the target position, one or more objects modeling an embedded circuit board (e.g., the circuit board 20) can be placed on the shell model, e.g., by the editor 234. The locations of the objects placed on the shell model can be automatically determined, e.g., by the locator 232. In some examples, positioning of the objects can mirror the placements of different parts of the circuit board and related components (e.g., the left arm 62, the right arm 64, the central leg 66, the power supply unit 28, the PPG sensor 38, etc.).

At step 318, the method can generate a modified teeth model which includes a dimple underneath the shell model at the target position. In some examples, the dimple can have a flat bottom and is sized to receive the sensor with a snap fit.

The modeling phase 310 outputs the modified teeth model, which can be used in the manufacturing phase 320 for producing the mouthpiece.

At step 322, a mold can be created (e.g., by the digital fabricator 250) based on the modified teeth model. The mold has a recessed portion corresponding to the dimple of the modified teeth model.

At step 324, a first elastomeric layer can be formed over the mold. The first elastomeric layer can have a protruded portion configured to be matingly received within the recessed portion of the mold. In some examples, the first elastomeric layer is substantially transparent.

At step 326, a circuit board including the sensor can be placed over the first elastomeric layer. Specifically, the sensor can be placed in a pocket formed by the protruded portion. In some examples, a flat top surface of the sensor is configured to maintain a flush contact with a flat bottom of the pocket.

At step 328, a second elastomeric layer can be formed over the first elastomeric layer and the circuit board. As a result, the circuit board, including the sensor, is sandwiched between the first and second elastomeric layers. At least a portion of the second elastomeric layer overlying the sensor is substantially opaque to prevent light from passing therethrough. In some examples, the whole or a substantial part of the second elastomeric layer is opaque so that any ambient light entering the user's mouth is blocked by the second elastomeric layer.

At step 330, a third elastomeric layer can be formed over the second elastomeric layer. In some examples, labels carrying information and/or logos can be placed on the second elastomeric layer before forming the third elastomeric layer so that the labels are sandwiched between the second and third elastomeric layers. In some cases, forming the third elastomeric layer can be optional.

The method 300 described in the flowchart of FIG. 3 and any of the other methods described herein can be performed by computer-executable instructions (e.g., causing a computing system to perform the method) stored in one or more computer-readable media (e.g., storage or other tangible media) or stored in one or more computer-readable storage devices. Such methods can be performed in software, firmware, hardware, or combinations thereof. Such methods can be performed at least in part by a computing system (e.g., one or more computing devices).

The illustrated actions can be described from alternative perspectives while still implementing the technologies. For example, “send” can also be described as “receive” from a different perspective.

### Example Manufacturing Process

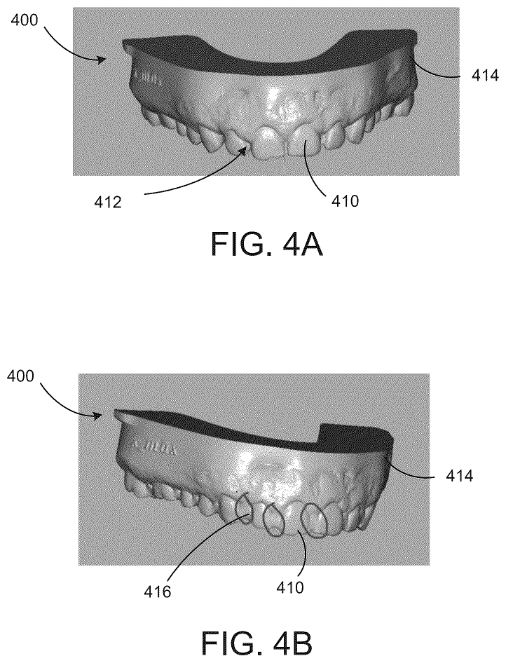

The method 300 described above can be further illustrated in FIGS. 4A-8C, which depicts various stages of the process for manufacturing a mouthpiece.

FIG. 4A shows a computer-rendered teeth model 400 (e.g., generated by the model renderer 226), which can be used to manufacture a mouthpiece. Preprocessing can be performed to identify individual teeth 410, gum 414, and gaps 412 between adjacent teeth 410 (e.g., by the segmentation unit 224).

FIG. 4B shows the teeth model 400 (slightly tilted compared to FIG. 4A) in which the identified gaps 412 between adjacent teeth 410 are filled with virtual fillers 416 (e.g., by the gap filler 228), as marked by the circles.

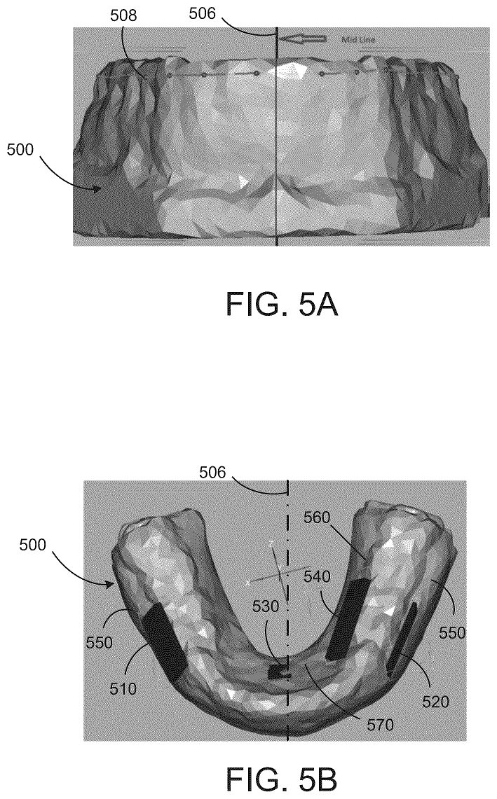

FIG. 5A shows a shell model 500 created (e.g., by the shell generator 230) based on the gap-filled teeth model 400 depicted in FIG. 4B. Because there are no gaps between adjacent teeth, the shell model 500 can have a relatively smooth and continuous outer surface.

A circuit board (e.g., the circuit board 20) embedded in the mouthpiece can have several spatially distributed parts (connected by a flexible substrate), such as a left arm, a right arm, a central leg, and a power supply unit connected to one of the parts, as depicted in FIG. 1B. Each part can be modeled by a corresponding object. For example, FIG. 5B shows multiple objects representing different parts or components of a circuit board that are placed on the shell model 500 (e.g., by the editor 234). As shown, a first object 510 can model a module or components included on the left arm, a second object 520 can model a module or components included on the right arm, a third object 530 can model a module or components (e.g., the PPG sensor) included on the central leg, a fourth object 540 can model the power supply unit, etc.

Positions and orientations of the objects (e.g., 510, 520, 530, 540, etc.) on the shell model 500 can be automatically determined (e.g., by the locator 232) based on parameters of the mouthpiece model, which can specify size and shape of the different parts of the circuit board, as well as positions of those parts relative to the inner layer of the mouthpiece.

For example, as shown in FIG. 5A, a midline 506 of the shell model 500 which bisects the shell model 500 (e.g., into a left half and a right half) can be identified. An upper edge of the circuit board (e.g., top edges of the left and right arms) can be aligned with a circumferential line 508 having a predefined vertical distance to the top surface of the shell model 500. In some examples, the circumferential line 508 can represent the top or occlusal surface of the teeth model 400.

Different objects can be placed in reference to the midline 506 and/or the circumferential line 508. For example, both the first object 510 and second object 520 can be placed on a front surface 550 (also referred to as “facial surface”) of the shell model 500 but positioned on opposite sides of the midline 506. Circumferential distances between the first and second objects 510, 520 and the midline 506 can be specified by parameters of the mouthpiece. The fourth object 540 can be placed on a lingual surface 560 of the shell model 500 (e.g., opposite to the second object 520). The third object 530 can be aligned with the midline 506 and placed on a palate surface 570 of the shell model 500.

In some examples, the position of the third object 530 generally corresponds to a target position of the PPG sensor located on the central leg. The palate surface 570 of the shell model 500 substantially matches at least a portion of the user's palate, which can be included in the teeth model 400. Depending on the shape of the user's palate, the palate surface 570 can have generally flat portions and some curved portions. In some examples, the target position of the PPG sensor can be located at a generally flat portion of the palate surface 570 along the midline 506 of the shell model 500. As described above, the central leg of the circuit board can have a flexible substrate. Thus, the PPG sensor located on the central leg can have a certain degree of freedom to be moved relative to the palate surface 570 so that it can be placed at the target position.

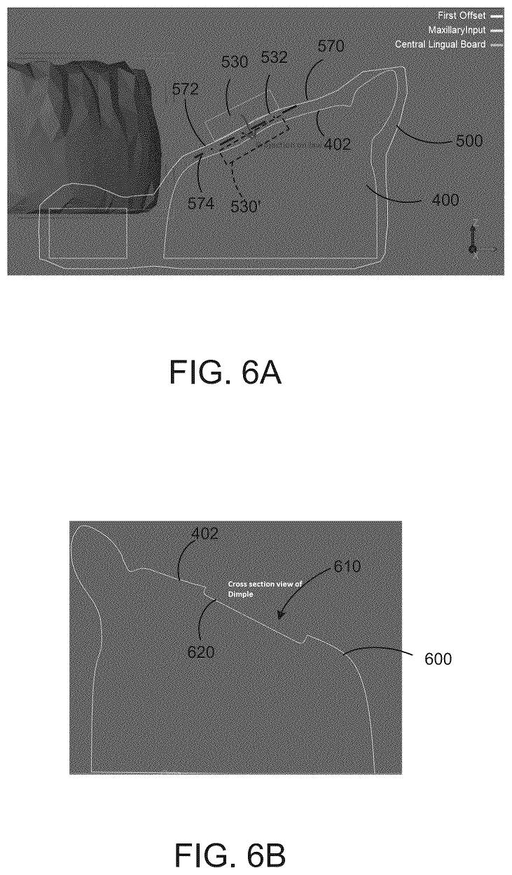

FIG. 6A shows a cross-sectional view of the shell model 500 and the underlying teeth model 400 taken along the midline 506. In the depicted example, the space between the palate surface 570 and an outer surface 402 of the teeth model 400 represents a thickness of the shell model 500. The third object 530 representing the PPG sensor can have a generally flat outer surface 532 sitting on the palate surface 570 of the shell model 500. As shown, the PPG sensor can be placed over a generally flat portion 572 of the palate surface 570, which sits on top of a generally flat portion of the outer surface 402 of the teeth model 400. Thus, the outer surface 532 of the PPG sensor can seamlessly contact the generally flat portion 572 of the palate surface 570.

The third object 530 representing the PPG sensor can be projected back (downwardly as indicated by the arrow in FIG. 6A) toward the teeth model 400 to create a mirror object 530′ (having the same shape and size as the third object 530) underneath the palate surface 570. The third object 530 and the mirror object 530′ can be symmetric about an axis 574 that is parallel to the generally flat portion 572. In some examples, the axis 574 can be located on a plane extending along the palate surface 570 of the shell model (e.g., the outer side of the shell model that does not directly contact the outer surface 402 of the teeth model). In some examples, the axis 574 can be on a plane extending over an inner surface of the shell model (e.g., the inner side of the shell model that contacts the outer surface 402 of the teeth model). In some examples, the axis 574 can be on a plane extending through a thickness of the shell model (e.g., between the palate surface 570 of the shell model and the outer surface 402 of the teeth model).

As described herein, a portion of the teeth model 400 overlapping with the mirror object 530′ can be removed (e.g., by the editor 234), resulting a modified teeth model 600, as shown in FIG. 6B. The shell model 500 is omitted from FIG. 6B for clarify.

As shown in FIG. 6B, the modified teeth model 600 includes a dimple 610 corresponding to the removed portion which overlaps with the mirror object 530′. The dimple 610 defines a void space underneath the shell model 500. For example, while the outer surface 402 of the teeth model outside the dimple 610 maintains contact with the shell model 500, a bottom surface 620 of the dimple 610 is spaced away from the shell model 500.

In some examples, the bottom surface 620 of the dimple 610 is flat or substantially flat to match the flatness of the outer surface 532 of the PPG sensor. In some examples, the dimple 610 is sized to receive the PPG sensor with a snap fit (e.g., the bottom surface 620 of the dimple 610 can have about the same shape and size as the outer surface 532 of the PPG sensor).

The depth of the dimple 610 can vary depending on where the axis 574 is located. For example, when the axis 574 is located on a plane extending over the inner surface of the shell model, the depth of the dimple 610 can be the same or substantially the same as a thickness of the PPG sensor. On the other hand, when the axis 574 is located a plane extending through a thickness or along the palate surface of the shell model, the depth of the dimple 610 can be a fraction of the thickness of the PPG sensor. For example, the dimple 610 schematically depicted in FIG. 6B is smaller than the thickness of the PPG sensor depicted in FIG. 6A. In some examples, the depth of the dimple 610 can be configured by the operator through the user interface 222. For example, through the user interface 222, the operator can set the depth of the dimple 610 to a fixed value, or the operator can configure the dimple's depth as a predefined percentage (greater than 0% and up to 100%) of the thickness of the PPG sensor. For example, the predefined percentage can be 10%, 20%, 30%, 40%, 50%, 60%, 70%, 80%, 90%, 100%, etc. In some examples, the depth of the dimple 610 can be adjusted to account for the curvature or variation of the outer surface 402 of the teeth model, ensuring that the dimple 610 is properly recessed from the outer surface 402, regardless of its shape.

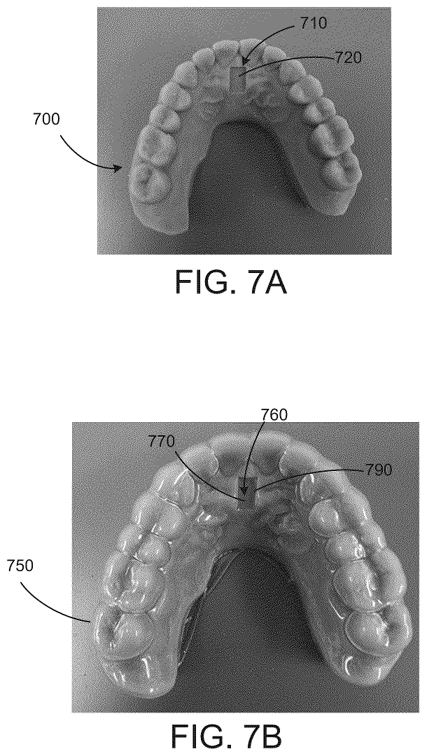

FIG. 7A shows a mold 700 which can be created (e.g., by the digital fabricator 250) based on the modified teeth model 600. As shown, the mold 700 has a recessed portion 710 corresponding to the dimple 610 of the modified teeth model 600. For example, the recessed portion 710 can have a flat or substantially flat bottom 720 corresponding to the bottom surface 620 of the dimple 610.

FIG. 7B shows a first elastomeric layer 750 formed (e.g., by the thermoforming apparatus 270) over the mold 700. In some examples, the first elastomeric layer 750 includes EVA plastic. The first elastomeric layer 750 has a protruded portion 790 configured to be matingly received within the recessed portion 710 of the mold 700. The protruded portion 790 can form a pocket 760 which has a flat or substantially flat bottom 770 matching the flat bottom 720 of the recessed portion 710 of the mold 700.

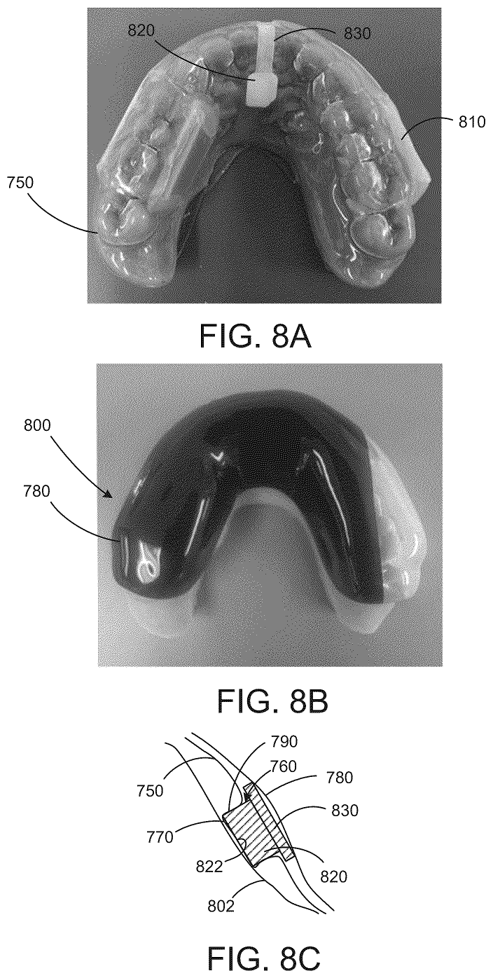

FIG. 8A shows a circuit board 810 being placed over the first elastomeric layer 750. The circuit board 810 has a flexible substrate including a central leg 830 with an integrated PPG sensor 820. As shown in FIGS. 8A and 8C, the PPG sensor 820 can be retained in the pocket 760. The pocket 760 can be shaped and sized to receive the PPG sensor 820 with a snap fit.

As shown in FIG. 8A, the first elastomeric layer 750 can be substantially transparent to allow light to pass therethrough. In some examples, only selected portions of first elastomeric layer 750 are transparent. For example, portions of the first elastomeric layer overlying a light source (e.g., the light source 32) can be transparent to allow the light emitted from the light source to shine therethrough and onto the user's palate, and the protruded portion 790 of the first elastomeric layer 750 can also be transparent to allow the light reflected back from the user's palate to pass therethrough and detected by the PPG sensor 820 received within the pocket 760.

FIG. 8B show a manufactured mouthpiece 800 which includes a second elastomeric layer 780 formed (e.g., by the thermoforming apparatus 270) over the first elastomeric layer 750 and the circuit board 810. In some examples, the second elastomeric layer 780 includes EVA plastic. The circuit board 810, sandwiched between the first elastomeric layer 750 and the second elastomeric layer 780, can be firmly held in place due to the adhesive properties of the EVA plastic when thermoformed.

As shown in FIG. 8B, a majority part of the second elastomeric layer 780, including the middle portion which covers the PPG sensor 820, is substantially opaque so as to prevent ambient light entering the user's mouth from being detected by the PPG sensor 820. In some examples, the whole second elastomeric layer 780 can be opaque.

The depths of the pocket 760 can vary depending on the depth of the dimple 610 on the modified teeth model 600 (which also defines the depth of the recessed portion 710 on the mold 700). In the example depicted in FIG. 8C, the depth of the pocket 760 substantially matches a thickness of the PPG sensor 820 so that the PPG sensor 820 is approximately completely inserted into the pocket 760. In other examples, the depth of the pocket 760 can be a fraction of the thickness of the PPG sensor 820 so that a portion of the PPG sensor 820 is inserted into the pocket 760 whereas the remaining portion of the PPG sensor 820 can extend outside the pocket 760.

Generally, the PPG sensor 820 can have a flat or substantially flat outer surface 822 which maintains a seamless contact with the flat bottom 770 of the pocket 760, as illustrated in FIG. 8C. Because the location of the dimple 610 on the modified teeth model 600 corresponds to the generally flat portion 572 of the palate surface 570 (FIGS. 6A-6B) which models the user's palate 802, the flat bottom 770 of the resulting pocket 760 also sits on top of a flat surface of the user's palate 802. As a result, the PPG sensor 820 ensures a stable and consistent contact with the user's palate 802, providing accurate and reliable readings by minimizing any potential movement or misalignment during use.

### Example Advantages

The disclosed technologies present several technical advantages that address the challenges of existing mouthpiece manufacturing processes. One significant improvement is the clear identification of the location for the placement of PPG sensors. This is achieved through accurate mapping of the user's palate topology, and strategically placing a dimple on the user's teeth model. This dimple corresponds to a flat portion of the user's palate, ensuring that the PPG sensor maintains stable contact for reliable PPG signal measurement. The automated process eliminates the need for manual sensor placement, thereby reducing human error and increasing production efficiency. Further, the customization of user's teeth model with the dimple ensures that the PPG sensor embedded in the mouthpiece can apply a consistent pressure to the user's palate, ensuring not only more accurate and stable readings of PPG signals, but also user comfort.

### Example Computing Systems

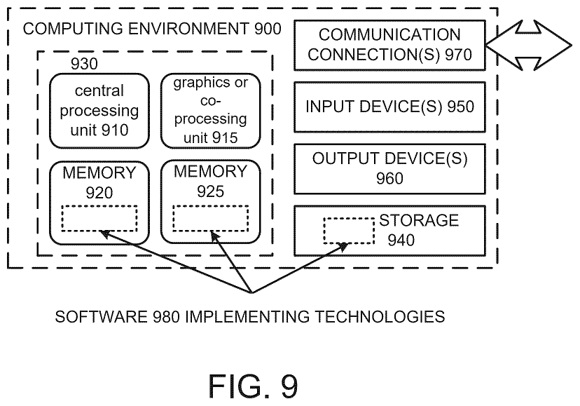

FIG. 9 depicts an example of a suitable computing system 900 in which the described innovations can be implemented. For example, the computing system 900 can be used in the modeling engine 220 and/or the digital fabricator 250 depicted in FIG. 2. The computing system 900 is not intended to suggest any limitation as to scope of use or functionality of the present disclosure, as the innovations can be implemented in diverse computing systems.

With reference to FIG. 9, the computing system 900 includes one or more processing units 910, 915 and memory 920, 925. In FIG. 9, this basic configuration 930 is included within a dashed line. The processing units 910, 915 execute computer-executable instructions, such as for implementing the features described in the examples herein. A processing unit can be a general-purpose central processing unit (CPU), processor in an application-specific integrated circuit (ASIC), or any other type of processor. In a multi-processing system, multiple processing units execute computer-executable instructions to increase processing power. For example, FIG. 9 shows a central processing unit 910 as well as a graphics processing unit or co-processing unit 915. The tangible memory 920, 925 can be volatile memory (e.g., registers, cache, RAM), non-volatile memory (e.g., ROM, EEPROM, flash memory, etc.), or some combination of the two, accessible by the processing unit(s) 910, 915. The memory 920, 925 stores software 980 implementing one or more innovations described herein, in the form of computer-executable instructions suitable for execution by the processing unit(s) 910, 915.

A computing system 900 can have additional features. For example, the computing system 900 includes storage 940, one or more input devices 950, one or more output devices 960, and one or more communication connections 970, including input devices, output devices, and communication connections for interacting with a user. An interconnection mechanism (not shown) such as a bus, controller, or network interconnects the components of the computing system 900. Typically, operating system software (not shown) provides an operating environment for other software executing in the computing system 900, and coordinates activities of the components of the computing system 900.

The tangible storage 940 can be removable or non-removable, and includes magnetic disks, magnetic tapes or cassettes, CD-ROMs, DVDs, or any other medium which can be used to store information in a non-transitory way and which can be accessed within the computing system 900. The storage 940 stores instructions for the software implementing one or more innovations described herein.

The input device(s) 950 can be an input device such as a keyboard, mouse, pen, or trackball, a voice input device, a scanning device, touch device (e.g., touchpad, display, or the like) or another device that provides input to the computing system 900. The output device(s) 960 can be a display, printer, speaker, CD-writer, or another device that provides output from the computing system 900.

The communication connection(s) 970 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier.

The innovations can be described in the context of computer-executable instructions, such as those included in program modules, being executed in a computing system on a target real or virtual processor (e.g., which is ultimately executed on one or more hardware processors). Generally, program modules or components include routines, programs, libraries, objects, classes, components, data structures, etc. that perform particular tasks or implement particular abstract data types. The functionality of the program modules can be combined or split between program modules as desired in various examples. Computer-executable instructions for program modules can be executed within a local or distributed computing system.

For the sake of presentation, the detailed description uses terms like “determine” and “use” to describe computer operations in a computing system. These terms are high-level descriptions for operations performed by a computer and should not be confused with acts performed by a human being. The actual computer operations corresponding to these terms vary depending on implementation.

### Example Computer-Readable Media

Any of the computer-readable media herein can be non-transitory (e.g., volatile memory such as DRAM or SRAM, nonvolatile memory such as magnetic storage, can be implemented by storing in one or more computer-readable media (e.g., computer-readable storage media or other tangible media). Any of the things (e.g., data created and used during implementation) described as stored can be stored in one or more computer-readable media (e.g., computer-readable storage media or other tangible media). Computer-readable media can be limited to implementations not consisting of a signal.

Any of the methods described herein can be implemented by computer-executable instructions in (e.g., stored on, encoded on, or the like) one or more computer-readable media (e.g., computer-readable storage media or other tangible media) or one or more computer-readable storage devices (e.g., memory, magnetic storage, optical storage, or the like). Such instructions can cause a computing device to perform the method. The technologies described herein can be implemented in a variety of programming languages.

### General Considerations

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art. Any theories of operation are to facilitate explanation, but the disclosed systems, methods, and apparatus are not limited to such theories of operation.

As used in this application and in the claims, the singular forms “a,” “an,” and “the” include the plural forms unless the context clearly dictates otherwise. Additionally, the term “includes” means “comprises.” Further, the terms “coupled” and “connected” generally mean electrically, electromagnetically, and/or physically (e.g., mechanically or chemically) coupled or linked and does not exclude the presence of intermediate elements between the coupled or associated items absent specific contrary language.

Directions and other relative references (e.g., inner, outer, upper, lower, etc.) may be used to facilitate discussion of the drawings and principles herein, but are not intended to be limiting. For example, certain terms may be used such as “inside,” “outside,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

As used herein, “and/or” means “and” or “or,” as well as “and” and “or.”

### Example Clauses

Any of the following example clauses can be implemented.

Clause 1. A method for manufacturing a mouthpiece, the method comprising: receiving a digital representation of a teeth model; creating a shell model overlying an outer surface of the teeth model; identifying a target position on the shell model where a sensor of the mouthpiece is to be placed; and generating a modified teeth model comprising a dimple underneath the shell model at the target position.

Clause 2. The method of clause 1, wherein creating the shell model comprises filling one or more gaps between adjacent teeth in the teeth model.

Clause 3. The method of any one of clauses 1-2, wherein identifying the target position on the shell model comprises determining a flat surface along a midline of the shell model.

Clause 4. The method of any one of clauses 1-3, further comprising creating a mold based on the modified teeth model, wherein the mold has a recessed portion corresponding to the dimple of the modified teeth model.

Clause 5. The method of clause 4, further comprising forming a first elastomeric layer over the mold, wherein the first elastomeric layer comprises a protruded portion configured to be matingly received within the recessed portion of the mold.

Clause 6. The method of clause 5, further comprising placing a circuit board including the sensor over the first elastomeric layer and forming a second elastomeric layer over the first elastomeric layer and the circuit board, wherein the sensor is placed in a pocket formed by the protruded portion.

Clause 7. The method of clause 6, wherein the first elastomeric layer is substantially transparent, and wherein at least a portion of the second elastomeric layer overlying the sensor is substantially opaque.

Clause 8. The method of any one of clauses 1-7, wherein the dimple has a flat bottom and is sized to receive the sensor with a snap fit.

Clause 9. A system for manufacturing a mouthpiece, the system comprising: memory; one or more hardware processors coupled to the memory; and one or more computer readable storage media storing instructions that, when loaded into the memory, cause the one or more hardware processors to perform operations comprising: receiving a digital representation of a teeth model; creating a shell model overlying an outer surface of the teeth model; identifying a target position on the shell model where a sensor of the mouthpiece is to be placed; and generating a modified teeth model comprising a dimple underneath the shell model at the target position.

Clause 10. The system of clause 9, wherein creating the shell model comprises filling one or more gaps between adjacent teeth in the teeth model.

Clause 11. The system of any one of clauses 9-10, wherein identifying the target position on the shell model comprises determining a flat surface along a midline of the shell model.

Clause 12. The system of any one of clauses 9-11, further comprising a digital fabricator configured to create a mold based on the modified teeth model, wherein the mold has a recessed portion corresponding to the dimple of the modified teeth model.

Clause 13. The system of clause 12, further comprising a thermoforming apparatus configured to form a first elastomeric layer over the mold, wherein the first elastomeric layer comprises a protruded portion configured to be matingly received within the recessed portion of the mold.

Clause 14. The system of clause 13, wherein the thermoforming apparatus is further configured to form a second elastomeric layer over the first elastomeric layer, wherein a circuit board including the sensor is configured to be sandwiched between the first elastomeric layer and the second elastomeric layer, wherein the sensor is placed in a pocket formed by the protruded portion.

Clause 15. The system of any one of clauses 9-14, wherein the dimple has a flat bottom and is sized to receive the sensor with a snap fit.

Clause 16. A mouthpiece, comprising: a first elastomeric layer configured to be placed over a user's teeth and gum; a second elastomeric layer covering the first elastomeric layer; and an electronic circuit sandwiched between the first elastomeric layer and the second elastomeric layer, wherein the first elastomeric layer comprises a protruded portion configured to press against a palate of the user when the mouthpiece is worn by the user, and wherein the electronic circuit comprises a sensor placed in a pocket formed by the protruded portion.

Clause 17. The mouthpiece of clause 16, wherein the sensor is a photoplethysmography sensor.

Clause 18. The mouthpiece of any one of clauses 16-17, wherein the pocket has a flat bottom and is sized to receive the sensor with a snap fit.

Clause 19. The mouthpiece of any one of clauses 16-18, wherein the first elastomeric layer is substantially transparent, and wherein at least a portion of the second elastomeric layer covering the sensor is substantially opaque.

Clause 20. The mouthpiece of any one of clauses 16-19, further comprising a third elastomeric layer covering the second elastomeric layer.

### Example Alternatives

The technologies from any example can be combined with the technologies described in any one or more of the other examples. In view of the many possible examples to which the principles of the disclosed technology can be applied, it should be recognized that the illustrated embodiments are examples of the disclosed technology and should not be taken as a limitation on the scope of the disclosed technology. Rather, the scope of the claimed subject matter is defined by the following claims and their equivalents.

What is claimed is:

1. A method for manufacturing a mouthpiece, the method comprising:
receiving a digital representation of a teeth model;
creating a shell model overlying an outer surface of the teeth model;
identifying a target position on the shell model where a sensor of the mouthpiece is to be placed; and
generating a modified teeth model comprising a dimple underneath the shell model at the target position.

2. The method of claim 1, wherein creating the shell model comprises filling one or more gaps between adjacent teeth in the teeth model.

3. The method of claim 1, wherein identifying the target position on the shell model comprises determining a flat surface along a midline of the shell model.

4. The method of claim 1, further comprising creating a mold based on the modified teeth model, wherein the mold has a recessed portion corresponding to the dimple of the modified teeth model.

5. The method of claim 4, further comprising forming a first elastomeric layer over the mold, wherein the first elastomeric layer comprises a protruded portion configured to be matingly received within the recessed portion of the mold.

6. The method of claim 5, further comprising placing a circuit board including the sensor over the first elastomeric layer and forming a second elastomeric layer over the first elastomeric layer and the circuit board, wherein the sensor is placed in a pocket formed by the protruded portion.

7. The method of claim 6, wherein the first elastomeric layer is substantially transparent, and wherein at least a portion of the second elastomeric layer overlying the sensor is substantially opaque.

8. The method of claim 1, wherein the dimple has a flat bottom and is sized to receive the sensor with a snap fit.

9. A system for manufacturing a mouthpiece, the system comprising:
memory;
one or more hardware processors coupled to the memory; and
one or more computer readable storage media storing instructions that, when loaded into the memory, cause the one or more hardware processors to perform operations comprising:
receiving a digital representation of a teeth model;
creating a shell model overlying an outer surface of the teeth model;
identifying a target position on the shell model where a sensor of the mouthpiece is to be placed; and
generating a modified teeth model comprising a dimple underneath the shell model at the target position.

10. The system of claim 9, wherein creating the shell model comprises filling one or more gaps between adjacent teeth in the teeth model.

11. The system of claim 9, wherein identifying the target position on the shell model comprises determining a flat surface along a midline of the shell model.

12. The system of claim 9, further comprising a digital fabricator configured to create a mold based on the modified teeth model, wherein the mold has a recessed portion corresponding to the dimple of the modified teeth model.

13. The system of claim 12, further comprising a thermoforming apparatus configured to form a first elastomeric layer over the mold, wherein the first elastomeric layer comprises a protruded portion configured to be matingly received within the recessed portion of the mold.

14. The system of claim 13, wherein the thermoforming apparatus is further configured to form a second elastomeric layer over the first elastomeric layer, wherein a circuit board including the sensor is configured to be sandwiched between the first elastomeric layer and the second elastomeric layer, wherein the sensor is placed in a pocket formed by the protruded portion.

15. The system of claim 9, wherein the dimple has a flat bottom and is sized to receive the sensor with a snap fit.

16. A mouthpiece, comprising:
a first elastomeric layer configured to be placed over a user's teeth and gum;
a second elastomeric layer covering the first elastomeric layer; and
an electronic circuit sandwiched between the first elastomeric layer and the second elastomeric layer,
wherein the first elastomeric layer comprises a protruded portion configured to press against a palate of the user when the mouthpiece is worn by the user, and
wherein the electronic circuit comprises a sensor placed in a pocket formed by the protruded portion.

17. The mouthpiece of claim 16, wherein the sensor is a photoplethysmography sensor.

18. The mouthpiece of claim 16, wherein the pocket has a flat bottom and is sized to receive the sensor with a snap fit.

19. The mouthpiece of claim 16, wherein the first elastomeric layer is substantially transparent, and wherein at least a portion of the second elastomeric layer covering the sensor is substantially opaque.

20. The mouthpiece of claim 16, further comprising a third elastomeric layer covering the second elastomeric layer.
